(19) United States
(12) Patent Application Publication
(10) Pub. No.: US 2005/0250752 A1
(43) Pub. Date: Nov. 10, 2005

(54) Cembranoids with chemopreventive activity

(76) Inventors: Jordan K. Ziawiony, Oxford, MS (US); Hesham Fahmy, Brookings (SD); Isamu Katsuyama, Ibaraki-shi (JP); Sherief I. Khalifa, Ismailia (EG); Takao Konoshima, Choshi City (JP)

Correspondence Address: STITES & HARBISON PLLC, 424 CHURCH STREET, SUITE 1800, NASHVILLE, TN, 37219-2376, US

(21) Appl. No.: 10/513,955

(22) PCT Filed: May 9, 2003
(86) PCT No.: PCT/US03/14666

§ 371 Date: Jun. 24, 2005

Related U.S. Application Data
(60) Provisional application No. 60/379,065, filed on May 9, 2002.

(51) Int. Cl. A61K 31/336; A61K 31/38; A61K 31/045

(52) U.S. Cl. 514/183; 514/729; 548/954; 549/90; 549/560; 568/821; 514/475; 514/430

(57) ABSTRACT

A cembranoid compound of the following formula (I) as described herein. The compounds of the present invention are useful as chemopreventive and chemotherapeutic agents.

### Priority Claim

This Application claims priority to U. S. Application Ser. No. 60/379,065, filed May 9, 2002.

### GOVERNMENT SUPPORT

This work is a result of research sponsored in part by the National Oceanic and Atmospheric Administration, Department of Commerce under Grants # NA56RG0129 and NA86RG0039, the Mississippi-Alabama Sea Grant Consortium, and Grant No. BIO4-002-002 awarded by the U.S.-Egypt Science and Technology Joint Fund in cooperation with the U.S. Department of Agriculture. The United States Government has certain rights in this invention.

### TECHNICAL FIELD OF THE INVENTION

The present invention relates to the field of chemopreventive agents. Furthermore, this invention relates to methods and compositions for preventing and/or treating cancer. Included in the methods for preventing cancer are methods for suppressing and inhibiting cancer, and methods for reducing the risk of developing cancer.

### BACKGROUND OF THE INVENTION

There is a demand for a chemopreventive drug in the marketplace. A number of natural products that show promise as anticancer drugs come from the marine environment. An example is sarcophytol A, an cembranoid isolated from the soft coral Sarcophytum glaucum. Sarcophytol A is known to suppress carcinogenesis. It especially inhibits the development of large bowel cancer and suppresses carcinogenesis in liver, breast, thymus, and skin.

Cembranoids are diterpenoids with a fourteen-membered ring that have been isolated from terrestrial and marine sources. The hydroxylated cembranoids sarcophytol A (1, below) and B (2, below) from the Okinawan soft coral Sarcophyton glaucum have attracted great attention because of their reported antitumor activity and inhibitory activity against tumor promoters.

Sarcophine (3), is an abundant cembranolide isolated from the Red Sea soft coral Sarcophyton glaucum. It is a fish toxin that is the chemical defense system against natural predators. Sarcophine acts as an inhibitor of a number of vital enzymes including cholinesterase, and phosphofructokinase.

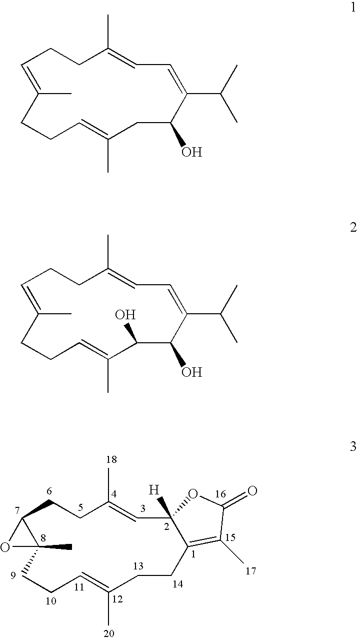

The present inventors, during studies on semisynthetic modification of sarcophine, discovered novel compounds with high inhibitory activity against antigens, including, for example, Epstein-Barr virus early antigen (EBV-EA) of Raji cells induced by tumor promoter (TPA). The compounds of the present invention exhibit an improved inhibitory effect on EBA-EA activation induced by TPA (including 92-96% inhibition at concentration of 32 μl/ml for some embodiments) as compared to sarcophytol A (88% inhibition at the same concentration).

To more fully describe the state of the art to which this invention pertains, the following references are provided:
Fujiki et al., Jpn. J. Cancer Chenother., 1986, 13, 3384-3391.
Fujiki et al., Basic Life Sci., 1990, 52, 205-212.
Muto et al., Jpn. J. Clin. Oncol., 1990, 20, 219-224.
Fujiki et al., Cancer Chemoprev., 1992, 393-405.
Fujiki et al., ACS Symp. Ser., 1992, 507 (Phenolic Compd. Food Their Effect Health II), 380-387.
Komori et al., Cancer Res., 1993, 53, 3462, 3464.
Bhimani et al., Cancer Res., 1993, 53, 4528-4533.
Fujiki et al., Cancer Detect. Prev., 1994, 18, 1-7.
Yokomatsu et al., Pancreas, 1994, 9, 526-530.
Steele et al., J. Cell Biochem., 1994 (Suppl. 20), 32-54.

Yun et al., Ann N. Y Acad. Sci., 1999, 899 (Cancer Prevention), 157-192.
Weitberg et al., J Exp. Clin. Cancer Res., 1999, 18, 433-437.
Wahlberg, I.; Eklund, A. M. Prog. Chem. Org. Nat. Prod. 1992, 59, 141-224.
Faulkner, D. J. Nat. Prod. Rep. 1988, 5, 613-663.
Weinheimer, A. J.; Chang, C. W. J.; Matson, J. A. Fortschr. Chem. Org. Naturst. 1979, 36, 285-387.
Tius, M. A. Chem. Rev. 1988, 88, 719-732.
Tursch, B.; Braeckman, J. C.; Dolaze, D.; Kaisin, M. In Marine Natural Products: Chemical and Biological Perspectives; Scheuer, P. J., Ed.; Academic: New York, 1978; Vol. 2, pp. 247-296.
Kobayashi, M.; Nakagawa, T.; Mitsuhashi, H. Chem. Pharm. Bull. 1979, 27, 2382-2387.
Suganuma, M.; Okabe, S.; Sueoka, E.; Iida, N.; Komori, A.; Kim, S.; Fujiki, H. Cancer Res. 1996, 56, 3711-3715.
Yamauchi, O.; Omori, M.; Ninomiya, M.; Okuno, M.; Moriwaki, H.; Suganuma, M.; Fujiki, H.; Muto, Y. Jpn. J. Cancer Res. 1991, 82, 1234-1238.
Fujiki, H.; Suganuma, M.; Suguri, H.; Yoshizawa, S.; Takagi, K.; Kobayashi, M. J. Cancer Res. 1989, 49, 25-28.
Japanese patent 81 61317 and 81 61318 to Mitsubishi Kasei Corporation; Chem. Abstr. 1981, 95, 169547 and 169548.
Bernstein, J.; Shmeuli, U.; Zadock, E.; Kashman, Y.; Neeman, I. Tetrahedron 1974, 30, 2817-2824.
Erman, A.; Neeman, I. Toxicon 1977, 15, 207-215. Neeman, I,; Fishelson, L.; Kashman, Y. Toxicon 1974, 12, 593-598.
Rabjohn, N. Org. React., 1976, 24, 261-415.
Czarkie, D., Groweiss, A., Kashman, Y., Tetrahedron, 1985, 41, 1049-1056.
Kobayashi, M., Ishizaka, T., Miura, N., Mitsuhashi, H. Chem. Pharm. Bull. 1987, 35, 2314-2318.
El Sayed, K. A.; Hamann, M. T.; Wadding, C. A.; Jensen, C.; Lee, S. K.; Dunstan, C. A.; Pezzuto, J. M. J. Org. Chem. 1998, 63, 7449-7455.
Takasaki M., Konoshima T., Komatsu K., Tokuda H., Nishino H., Cancer Letters, 2000, 158, 53-59.
Morita et al., Anti-Solid Tumor Agents, U.S. Pat. No. 5,387,609, issued Feb. 7, 1995.
Umezu et al., Lysosome Liberation inhibitors and Histamine Release Inhibitors, U.S. Pat. No. 4,906,794.
Katsuyama I., Fahmy H., Zjawiony J. K., Khalifa S., Kilada R. W., Konoshima T., Takasaki M., Tokuda H., J. Nat. Prod. 2002, 65, 1809-1814.
Fahmy H., Zjawiony J. K., Khalifa S., Fronczek F., Acta. Cryst. 2003, C59, o85-o87.
Motohashi N; Yamagami C; Tokuda H; Konoshima T; Okuda Y; Okuda M; Mukainaka T; Nishino H; Saito Y. Inhibitory effects of dehydrozingerone and related compounds on 12-O-tetradecanoylphorbol-13-acetate induced Epstein-Barr virus early antigen activation. Cancer Letters, 1998, 134, 37-42.
Ito, Hideyuki; Miyake, Masateru; Nishitani, Eisei; Mori, Kazuko; Hatano, Tsutomu; Okuda, Takuo; Konoshima, Takao; Takasaki, Midori; Kozuka, Mutsuo; Mukainaka, Teruo; Tokuda, Harukuni; Nishino, Hoyoku; Yoshida, Takashi. Anti-tumor promoting activity of polyphenols from Cowania mexicana and Coleogyne ramosissima. Cancer Letters 1999, 143, 5-13.

### SUMMARY OF THE INVENTION

The present invention includes compounds of the following formula, which is presented below and in all cases in the present invention includes salts thereof:

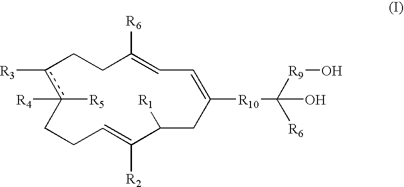

wherein:

R₁ is hydrogen, substituted or unsubstituted alkyl, hydroxyl, acyloxyl, or ketone;

R₂ is hydrogen, substituted or unsubstituted alkyl, hydroxymethyl, carboxaldehyde, or a carboxylic group;

R₃ is hydroxyl, alkoxyl, acyloxyl, amino, alkylamino, arylamino, arylalkylamino, Het-alkylamino, thio, alkylthio, arylthio, arylalkylthio, Het-alkylthio, or together with R₄ forms a double bond or a ring;

R₄ is hydroxyl, alkoxyl, acyloxyl, amino, alkylamino, arylamino, arylalkylamino, Het-alkylamino, thio, alkylthio, arylthio, arylalkylthio, Het-alkylthio, together with R₅ forms a double bond, or together with R₃ forms a double bond or a ring;

R₅ is hydrogen, substituted or unsubstituted alkyl group; or together with R₄ forms a double bond;

R₆ is H or substituted or unsubstituted alkyl group;

R₉ is a bond or a substituted or unsubstituted alkyl group; and

R₁₀ is a bond or a substituted or unsubstituted alkyl group;

with the proviso that when R₃ and R₄ form a double bond, R₂, R₅, R₆, and R₉ are not all methyl and at the same time R₁₀ is not a bond.

Embodiments of the present invention also include methods of preventing and/or treating cancer in a mammalian subject, comprising administering an effective amount to the mammalian subject a pharmaceutical preparation comprising a compound of the present invention and a pharmaceutical carrier.

Other embodiments of the present invention include methods of preparing the compounds of the present invention and methods of using the compounds of the present invention.

### DETAILED DESCRIPTION OF THE INVENTION

A. Compounds of the Present Invention

As stated above, compounds of the present invention can be represented by the following formula, which includes salts thereof:

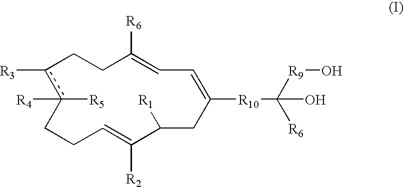

wherein:

R₁ is hydrogen, substituted or unsubstituted allyl, hydroxyl, acyloxyl, or ketone;

R₂ is hydrogen, substituted or unsubstituted alkyl, hydroxymethyl, carboxaldehyde, or a carboxylic group;

R₃ is hydroxyl, alkoxyl, acyloxyl, amino, alkylamino, arylamino, arylalkylamino, Het-alkylamino, thio, alkylthio, arylthio, arylalkylthio, Het-alkylthio, or together with R₄ forms a double bond or a ring;

R₄ is hydroxyl, alkoxyl, acyloxyl, amino, alkylamino, arylamino, arylalkylamino, Het-alkylamino, thio, alkylthio, arylthio, arylalkylthio, Het-alkylthio, together with R₅ forms a double bond, or together with R₃ forms a double bond or a ring;

R₅ is hydrogen, substituted or unsubstituted alkyl group; or together with R₄ forms a double bond;

R₆ is H or substituted or unsubstituted alkyl group;

R₉ is a bond or a substituted or unsubstituted alkyl group; and

R₁₀ is a bond or a substituted or unsubstituted alkyl group;

with the proviso that when R₃ and R₄ form a double bond, R₂, R₅, R₆, and R₉ are not all methyl and at the same time R₁₀ is not a bond.

Another example of a compound of the present invention is where R₁ is hydrogen; R₂ is a methyl group; R₃is a substituted or unsubstituted alkyl group, hydroxyl or methoxyl; and R₄is a substituted or unsubstituted alkyl group, hydroxyl or methoxyl. Alternatively, R₃ and R₄ form an oxirane ring.

With respect to the compounds described above, the alkyl portions of the R₃ and R₄ substituents may be a straight chain or branched alkyl having 1 to 4 carbon atoms. For example, the alkylamino group may be a butane group with an amino substituent. Further, the at least one of the aryl portions of the R₃ or R₄ substituent may be phenyl, or naphthyl. Specific examples of the Het portion include furyl, pyrrolyl, thienyl, imidazolyl, pyridinyl, pyridazinyl or pyrimidinyl.

Optionally, R₃ and R₄ may form a ring. Examples of this ring may be an aryl or Het group as defined herein. These rings may be optionally substituted with one or more substituents. Examples of the substituents include alkyl, alkoxy, perhaloalkyl, halogen, nitro, hydroxy, amino, carboxy, carboxyalkyl, alkylamino and dialkylamino, thioalkyl, alkoxycarbonyl and acyl. Examples of the rings formed by R₃and R₄ include 3-7 membered rings.

Further examples of rings formed by R₃ and R₄ include where R₃ and R₄ form an oxirane ring, thiirane ring, or a aziridine ring.

Another example of a compound of the present invention includes where, in formula (I), R₆ is H or substituted or unsubstituted alkyl; R₉ is methyl; and R₁₀ is a bond. Also, R₁ may be hydrogen; R₂ may be a methyl group; R₃ may be hydroxyl or methoxyl, or together with R₄ forms an oxirane or aziridine ring; R₄ may be hydroxyl or methoxyl, or together with R₃ forms an oxirane, thiirane, or aziridine ring; R₅ may be methyl; R₆may be H or substituted or unsubstituted alkyl; R₉ may be methyl; and R₁₀ may be a bond.

Additional compounds of the present invention include those of the following formula:

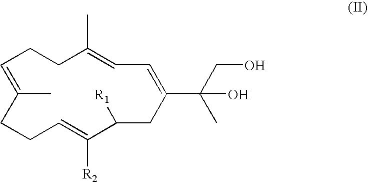

wherein R₁ and R₂ are as defined above, with the proviso that R₁ is not hydrogen when R₂ is methyl.

Compounds of the present invention include those of the following formula:

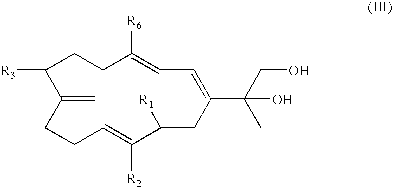

where R₁, R₂, and R₆ are defined above, and R₃ is hydroxyl, alkoxyl, acyloxyl, amino, alkylamino-, arylamino-, arylalkylamino, Het-alkylamino, thio, alkylthio, arylthio, arylalkylthio, or Het-arylalkylthio. Also with respect to this formula, R₁ may be hydrogen, R₂ may be a methyl group, R₃ may be hydroxyl or methoxyl, and R₆ may be a methyl group.

Additional embodiments include the following:

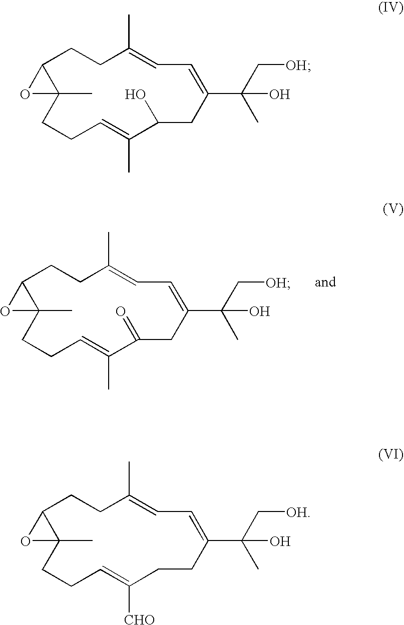

All the compounds of the present invention may be used in a chemopreventive or chemotherapeutic composition, comprising a compound of the present invention and a pharmaceutically acceptable carrier. This aspect of the invention is further discussed below.

As used herein, the term alkyl or alkyl group is to be understood in the broadest sense to mean hydrocarbon residues which can be linear, i.e., straight-chain, or branched, and can be acyclic or cyclic residues or comprise any combination of acyclic and cyclic subunits. Further, the term allyl as used herein expressly includes saturated groups as well as unsaturated groups which latter groups contain one or more, for example, one, two, or three, double bonds and/or triple bonds.

All these statements also apply if an alkyl group carries substituents or occurs as a substituent on another residue, for example, in an alkyloxy residue, or an arylalkylamino residue. Examples of alkyl residues containing from 1 to 20 carbon atoms are methyl, ethyl, propyl, butyl, pentyl, hexyl, heptyl, octyl, nonyl, decyl, undecyl, dodecyl, tetradecyl, hexadecyl, octadecyl, and eicosyl, the n-isomers of all these residues, isopropyl, isobutyl, 1-methylbutyl, isopentyl, neopentyl, 2,2-dimethylbutyl, 2-methylpentyl, 3-methylpentyl, isohexyl, 2,3,4-trimethylhexyl, isodecyl, sec-butyl, tert-butyl, or tert-pentyl.

Unsaturated alkyl residues are, for example, alkenyl residues such as vinyl, 1-propenyl, 2-propenyl (=allyl), 2-butenyl, 3-butenyl, 2-methyl-2-butenyl, 3-methyl-2-butenyl, 5-hexenyl, or 1,3-pentadienyl, or alkynyl residues such as ethynyl, 1-propynyl, 2-propynyl (=propargyl), or 2-butynyl. Alkyl residues can also be unsaturated when they are substituted.

Examples of cyclic alkyl residues are cycloalkyl residues containing 3, 4, 5, 6, 7, or 8 ring carbon atoms like cyclopropyl, cyclobutyl, cyclopentyl, cyclohexyl, cycloheptyl, or cyclooctyl which can also be substituted and/or unsaturated. Unsaturated cyclic alkyl groups and unsaturated cycloalkyl groups like, for example, cyclopentenyl or cyclohexenyl can be bonded via any carbon atom. The term alkyl as used herein also comprises cycloalkyl-substituted alkyl groups like cyclopropylmethyl-, cyclobutylmethyl-, cyclopentylmethyl-, cyclohexylmethyl-, cycloheptylmethyl-, cyclooctylmethyl-, 1-cyclopropylethyl-, 1-cyclobutylethyl-, 1-cyclopentylethyl-, 1-cyclohexylethyl-, 1-cycloheptylethyl-, 1-cyclooctylethyl-, 2-cyclopropylethyl-, 2-cyclobutylethyl-, 2-cyclopentylethyl-, 2-cyclohexylethyl-, 2-cycloheptylethyl-, 2-cyclooctylethyl-, 3-cyclopropylpropyl-, 3-cyclobutylpropyl-, 3-cyclopentylpropyl-, 3-cyclohexylpropyl-, 3-cycloheptylpropyl-, or 3-cyclooctylpropyl- in which groups the cycloalkyl subgroup as well as acyclic subgroup also can be unsaturated and/or substituted.

Of course, a group like (C₁-C₈)-alkyl is to be understood as comprising, among others, saturated acyclic (C₁-C₈)-alkyl, (C₃-C₈)-cycloalkyl, cycloalkyl-alkyl groups like (C₃-C₇)-cycloalkyl-(C₁-C₅)-alkyl- wherein the total number of carbon atoms can range from 4 to 8, and unsaturated (C₂-C₈)-alkyl like (C₂-C₈ )-alkenyl or (C₂-C₈)-alkynyl. Similarly, a group like (C₁-C₄)-alkyl is to be understood as comprising, among others, saturated acyclic (C₁-C₄)-alkyl, (C₃-C₄)-cycloalkyl, cyclopropyl-methyl-, and unsaturated (C₂-C₄)-alkyl like (C₂-C₄)-alkenyl or (C₂-C₄)-alkynyl.

Unless stated otherwise, the term alkyl preferably comprises acyclic saturated hydrocarbon residues containing from 1 to 6 carbon atoms which can be linear or branched, acyclic unsaturated hydrocarbon residues containing from 2 to 6 carbon atoms which can be linear or branched like (C₂-C₆)-alkenyl and (C₂-C₆)-alkynyl, and cyclic alkyl groups containing from 3 to 8 ring carbon atoms, in particular from 3 to 6 ring carbon atoms. A particular group of saturated acyclic alkyl residues is formed by (C₁-C₄)-alkyl residues like methyl, ethyl, n-propyl, isopropyl, n-butyl, isobutyl, sec-butyl, and tert-butyl.

The alkyl groups of the present invention can in general be unsubstituted or substituted by one or more, for example, one, two, three, or four, identical or different substituents. Any kind of substituents present in substituted alkyl residues can be present in any desired position provided that the substitution does not lead to an unstable molecule. Examples of substituted allyl residues are alkyl residues in which one or more, for example, 1, 2, 3, 4, or 5, hydrogen atoms are replaced with halogen atoms.

Examples of substituted cycloalkyl groups are cycloalkyl groups which carry as substituent one or more, for example, one, two, three, or four, identical or different acyclic alkyl groups, for example, acyclic (C₁-C₄)-alkyl groups like methyl groups. Examples of substituted cycloalkyl groups are 4-methylcyclohexyl, 4-tert-butylcyclohexyl, or 2,3-dimethylcyclopentyl.

The term aryl refers to a monocyclic or polycyclic hydrocarbon residue in which at least one carbocyclic ring is present. In a (C₆-C₁₄)-aryl residue from 6 to 14 ring carbon atoms are present. Examples of (C₆-C₁₄-aryl residues are phenyl, naphthyl, biphenylyl, fluorenyl, or anthracenyl. Examples of (C₆-C₁₀)-aryl residues are phenyl or naphthyl. Unless stated otherwise, and irrespective of any specific substituents bonded to aryl groups, aryl residues including, for example, phenyl, naphthyl, and fluorenyl, can in general be unsubstituted or substituted by one or more, for example, one, two, three, or four, identical or different substituents. Aryl residues can be bonded via any desired position, and in substituted aryl residues the substituents can be located in any desired position.

In monosubstituted phenyl residues, the substituent can be located in the 2-position, the 3-position, or the 4-position, the 3-position and the 4-position being preferred. If a phenyl group carries two substituents, they can be located in 2,3-position, 2,4-position, 2,5-position, 2,6-position, 3,4-position, or 3,5-position. In phenyl residues carrying three substituents, the substituents can be located in 2,3,4-position, 2,3,5-position, 2,3,6-position, 2,4,5-position, 2,4,6-position, or 3,4,5-position. Naphthyl residues can be 1-naphthyl and 2-naphthyl. In substituted naphthyl residues, the substituents can be located in any positions, for example, in monosubstituted 1-naphthyl residues in the 2-, 3-, 4-, 5-, 6-, 7-, or 8-position and in monosubstituted 2-naphthyl residues in the 1-, 3-, 4-, 5-, 6-, 7-, or 8-position. Biphenylyl residues can be 2-biphenylyl, 3-biphenylyl, or 4-biphenylyl. Fluorenyl residues can be 1-, 2-, 3-, 4-, or 9-fluorenyl. In monosubstituted fluorenyl residues, bonded via the 9-position the substituent is preferably present in the 1-, 2-, 3-, or 4-position.

Unless stated otherwise, substituents that can be present in substituted aryl groups are, for example, (C₁-C₈)-alkyl, in particular (C₁-C₄)-alkyl, such as methyl, ethyl, or tert-butyl, hydroxy, (C₁-C₈)-alkyloxy, in particular (C₁-C₄)-alkyloxy, such as methoxy, ethoxy, or tert-butoxy, methylenedioxy, ethylenedioxy, F, Cl, Br, I, cyano, nitro, trifluoromethyl, trifluoromethoxy, hydroxymethyl, formyl, acetyl, amino, mono- or di-(C₁-C₄)-alkylamino, ((C₁-C₄)-alkyl)carbonylamino like acetylamino, hydroxycarbonyl, ((C₁-C₄)-alkyloxy) carbonyl, carbamoyl, optionally substituted phenyl, benzyl optionally substituted in the phenyl group, optionally substituted phenoxy, or benzyloxy optionally substituted in the phenyl group.

The above statements relating to aryl groups correspondingly apply to divalent residues derived from aryl groups, i.e., to arylene groups like phenylene which can be unsubstituted or substituted 1,2-phenylene, 1,3-phenylene, or 1,4-phenylene, or naphthalene which can be unsubstituted or substituted 1,2-naphthalenediyl, 1,3-naphthalenediyl, 1,4-naphthalenediyl, 1,5-naphthalenediyl, 1,6-naphthalenediyl, 1,7-naphthalenediyl, 1,8-naphthalenediyl, 2,3-naphthalenediyl, 2,6-naphthalenediyl, or 2,7-naphthalenediyl.

The above statements also correspondingly apply to the aryl subgroup in arylalkyl-groups. Examples of arylalkyl-groups which can also be unsubstituted or substituted in the aryl subgroup as well as in the alkyl subgroup, are benzyl, 1-phenylethyl, 2-phenylethyl, 3-phenylpropyl, 4-phenylbutyl, 1-methyl-3-phenyl-propyl, 1-naphthylmethyl, 2-naphthylmethyl, 1-(1-naphthyl)ethyl, 1-(2-naphthyl)ethyl, 2-(1-naphthyl)ethyl, 2-(2-naphthyl)ethyl, or 9-fluorenylmethyl.

The “Het” group comprises groups containing 3, 4, 5, 6, 7, 8, 9, or 10 ring atoms in the parent monocyclic or bicyclic heterocyclic ring system. In monocyclic Het groups, the heterocyclic ring preferably is a 3-membered, 4-membered, 5-membered, 6-membered, or 7-membered ring, particularly preferably, a 5-membered or 6-membered ring. In bicyclic Het groups, preferably two fused rings are present, one of which is a 5-membered ring or 6-membered heterocyclic ring and the other of which is a 5-membered or 6-membered heterocyclic or carbocyclic ring, i.e., a bicyclic Het ring preferably contains 8, 9, or 10 ring atoms, more preferably 9 or 10 ring atoms.

Het comprises saturated heterocyclic ring systems which do not contain any double bonds within the rings, as well as mono-unsaturated and poly-unsaturated heterocyclic ring systems which contain one or more, for example, one, two, three, four, or five, double bonds within the rings provided that the resulting system is stable. Unsaturated rings may be non-aromatic or aromatic, i.e., double bonds within the rings in the Het group may be arranged in such a manner that a conjugated pi electron system results. Aromatic rings in a Het group may be 5-membered or 6-membered rings, i.e., aromatic groups in a Het group contain 5 to 10 ring atoms. Aromatic rings in a Het group thus comprise 5-membered and 6-membered monocyclic heterocycles and bicyclic heterocycles composed of two 5-membered rings, one 5-membered ring, and one 6-membered ring, or two 6-membered rings. In bicyclic aromatic groups in a Het group, one or both rings may contain heteroatoms. Aromatic Het groups may also be referred to by the customary term heteroaryl for which all the definitions and explanations above and below relating to Het correspondingly apply.

Unless stated otherwise, in the Het groups and any other heterocyclic groups, preferably 1, 2, 3, or 4 identical or different ring heteroatoms selected from nitrogen, oxygen, and sulfur are present. Particularly preferably, in these groups 1 or 2 identical or different ring heteroatoms selected from nitrogen, oxygen, and sulfur are present. The ring heteroatoms can be present in any desired number and in any position with respect to each other provided that the resulting heterocyclic system is known in the art and is stable and suitable as a subgroup in a drug substance. Examples of parent structures of heterocycles from which the Het group can be derived are aziridine, oxirane, thiirane, azetidine, pyrrole, furan, thiophene, dioxole, imidazole, pyrazole, oxazole, isoxazole, thiazole, isothiazole, 1,2,3-triazole, 1,2,4-triazole, tetrazole, pyridine, pyran, thiopyran, pyridazine, pyrimidine, pyrazine, 1,2-oxazine, 1,3-oxazine, 1,4-oxazine, 1,2-thiazine, 1,3-thiazine, 1,4-thiazine, 1,2,3-triazine, 1,2,4-triazine, 1,3,5-triazine, azepine, 1,2-diazepine, 1,3-diazepine, 1,4-diazepine, indole, isoindole, benzofuran, benzothiophene, 1,3-benzodioxole, indazole, benzimidazole, benzoxazole, benzothiazole, quinoline, isoquinoline, chromane, isochromane, cinnoline, quinazoline, quinoxaline, phthalazine, pyridoimidazoles, pyridopyridines, pyridopyrimidines, purine, or pteridine, as well as ring systems which result from the listed heterocycles by fusion (or condensation) of a carbocyclic ring, for example, benzo-fused, cyclopenta-fused, cyclohexa-fused, or cyclohepta-fused derivatives of these heterocycles.

The Het residue may be bonded via any ring carbon atom, and in the case of nitrogen heterocycles, via any suitable ring nitrogen atom. Thus, for example, a pyrrolyl residue can be 1-pyrrolyl, 2-pyrrolyl, or 3-pyrrolyl, a pyrrolidinyl residue can be 1-pyrrolidinyl (=pyrrolidino), 2-pyrrolidinyl, or 3-pyrrolidinyl, a pyridyl residue can be 2-pyridyl, 3-pyridyl, or 4-pyridyl, and a piperidinyl residue can be 1-piperidinyl (=piperidino), 2-piperidinyl, 3-piperidinyl, or 4-piperidinyl. Furyl can be 2-furyl or 3-furyl, thienyl can be 2-thienyl or 3-thienyl, imidazolyl can be 1-imidazolyl, 2-imidazolyl, 4-imidazolyl, or 5-imidazolyl, 1,3-oxazolyl can be 1,3-oxazol-2-yl, 1,3-oxazol-4-yl, or 1,3-oxazol-5-yl, 1,3-thiazolyl can be 1,3-thiazol-2-yl, 1,3-thiazol-4-yl, or 1,3-thiazol-5-yl, pyrimidinyl can be 2-pyrimidinyl, 4-pyrimidinyl (=6-pyrimidinyl), or 5-pyrimidinyl, and piperazinyl can be 1-piperazinyl (=4-piperazinyl=piperazino) or 2-piperazinyl. Indolyl can be 1-indolyl, 2-indolyl, 3-indolyl, 4-indolyl, 5-indolyl, 6-indolyl, or 7-indolyl. Similarly, benzimidazolyl, benzoxazolyl, and benzothiazolyl residues can be bonded via the 2-position and via any of the positions 4, 5, 6, and 7, benzimidazolyl also via the 1-position. Quinolyl can be 2-quinolyl, 3-quinolyl, 4-quinolyl, 5-quinolyl, 6-quinolyl, 7-quinolyl, or 8-quinolyl, and isoquinolyl can be 1-isoquinolyl, 3-isoquinolyl, 4-isoquinolyl, 5-isoquinolyl, 6-isoquinolyl, 7-isoquinolyl, or 8-isoquinolyl. In addition to being bonded via any of the positions indicated for quinolyl and isoquinolyl, 1,2,3,4-tetrahydroquinolyl and 1,2,3,4-tetrahydroisoquinolyl can also be bonded via the nitrogen atoms in the 1-position and 2-position, respectively.

Unless stated otherwise, and irrespective of any specific substituents bonded to Het groups or any other heterocyclic groups which are indicated in the definition of compounds of the present invention, the Het group can be unsubstituted or substituted on ring carbon atoms with one or more, for example, one, two, three, four, or five, identical or different substituents like (C₁-C₈)-alkyl, in particular (C₁-C₄)-alkyl, (C₁-C₈)-alkyloxy, in particular (C₁-C₄)-alkyloxy, (C₁-C₄)-alkylthio, halogen, nitro, amino, ((C₁-C₄)-alkyl)carbonylamino like acetylamino, trifluoromethyl, trifluoromethoxy, hydroxy, oxo, hydroxy-(C₁-C₄)-alkyl such as, for example, hydroxymethyl, 1-hydroxyethyl, or 2-hydroxyethyl, methylenedioxy, ethylenedioxy, formyl, acetyl, cyano, methylsulfonyl, hydroxycarbonyl, aminocarbonyl, (C₁-C₄)-alkyloxycarbonyl, optionally substituted phenyl, optionally substituted phenoxy, benzyl optionally substituted in the phenyl group, or benzyloxy optionally substituted in the phenyl group. The substituents can be present in any desired position provided that a stable molecule results. Of course an oxo group cannot be present in an aromatic ring. Each suitable ring nitrogen atom in a Het group can independently of each other be unsubstituted, i.e., carry a hydrogen atom, or can be substituted, i.e., carry a substituent like (C₁-C₈)-alkyl, for example, (C₁-C₄)-alkyl such as methyl or ethyl, optionally substituted phenyl, phenyl-(C₁-C₄)-alkyl, for example, benzyl, optionally substituted in the phenyl group, hydroxy-(C₂-C₄)-alkyl such as, for example, 2-hydroxyethyl, acetyl, or another acyl group, methylsulfonyl or another sulfonyl group, aminocarbonyl, or (C₁-C₄)-alkyloxycarbonyl. Nitrogen heterocycles can also be present as N-oxides or as quaternary salts. Ring sulfur atoms can be oxidized to the sulfoxide or to the sulfone. Thus, for example, a tetrahydrothienyl residue may be present as S,S-dioxotetrahydrothienyl residue or a thiomorpholinyl residue like 4-thiomorpholinyl may be present as 1-oxo-4-thiomorpholinyl or 1,1-dioxo-4-thiomorpholinyl. A substituted Het group that can be present in a specific position of compounds of formula I can independently of other Het groups be substituted by substituents selected from any desired subgroup of the substituents listed before and/or in the definition of that group.

The explanations relating to the Het residue correspondingly apply to divalent Het residues including divalent heteroaromatic residues which may be bonded via any two ring carbon atoms and in the case of nitrogen heterocycles via any carbon atom and any suitable ring nitrogen atom or via any two suitable nitrogen atoms. For example, a pyridinediyl residue can be 2,3-pyridinediyl, 2,4-pyridinediyl, 2,5-pyridinediyl, 2,6-pyridinediyl, 3,4-pyridinediyl, or 3,5-pyridinediyl, a piperidinediyl residue can be, among others, 1,2-piperidinediyl, 1,3-piperidinediyl, 1,4-piperidinediyl, 2,3-piperidinediyl, 2,4-piperidinediyl, or 3,5-piperidinediyl, and a piperazinediyl residue can be, among others, 1,3-piperazinediyl, 1,4-piperazinediyl, 2,3-piperazinediyl, or 2,5-piperazinediyl. The above statements also correspondingly apply to the Het subgroup in the Het-alkyl-groups. Examples of such Het-alkyl-groups which can also be unsubstituted or substituted in the Het subgroup as well as in the alkyl subgroup, are (2-pyridyl)methyl, (3-pyridyl)methyl, (4-pyridyl)methyl, 2-(2-pyridyl)ethyl, 2-(3-pyridyl)ethyl, or 2-(4-pyridyl)ethyl.

Alkoxy as used herein means an alkyl-O— group in which the alkyl group is as previously described. Exemplary alkoxy groups include methoxy, ethoxy, n-propoxy, i-propoxy, n-butoxy, t-butoxy and polyethers including —O—(CH₂)₂ OCH₃.

An acyl group is defined as a group —C(O)R where R is an alkyl or aryl radical and includes acetyl, trifluoroacetyl, benzoyl and the like.

Where terms are used in combination, the definition for each individual part of the combination applies unless defined otherwise. For instance, arylalkylthio refers to an aryl group, as defined above, alkyl group as defined above, and a thio group. An example is alkylamino, which is defined as a nitrogen atom substituted with an alkyl of 1 to 12 carbon atoms. Also, thioalkyl, or alkylthio as used herein means an alkyl-S-group in which the alkyl group is as previously described. Thioalkyl groups include thiomethyl and the like. Examples of alkylthio groups of compounds of the present invention includes those groups having one or more thioether linkages and from 1 to about 12 carbon atoms, further examples have from 1 to about 8 carbon atoms, and still further examples have 1 to about 6 carbon atoms. Alkylthio groups having 1, 2, 3 or 4 carbon atoms are further examples.

Some of the compounds of the invention may have stereogenic centers. The compounds may, therefore, exist in at least two and often more stereoisomeric forms. The present invention encompasses all stereoisomers of the compounds whether free from other stereoisomers or admixed with other stereoisomers in any proportion and thus includes, for instance, racemic mixture of enantiomers as well as the diastereomeric mixture of isomers. Thus, when using the term compound, it is understood that all stereoisomers are included.

The compounds of the present invention may be obtained or used as inorganic or organic salts using methods known to those skilled in the art. It is well known to one skilled in the art that an appropriate salt form is chosen based on physical and chemical stability, flowability, hydroscopicity and solubility. Pharmaceutically acceptable salts of the present invention with an acidic moiety may be optionally formed from organic and inorganic bases. For example with alkali metals or alkaline earth metals such as sodium, potassium, lithium, calcium, or magnesium or organic bases and N-tetraalkylammonium salts such as N-tetrabutylammonium salts. Similarly, when a compound of this invention contains a basic moiety, salts may be optionally formed from organic and inorganic acids.

For example salts may be formed from acetic, propionic, lactic, citric, tartaric, succinic, fumaric, maleic, malonic, mandelic, malic, phthalic, hydrochloric, hydrobromic, phosphoric, nitric, sulfuric, methanesulfonic, naphthalenesulfonic, benzenesulfonic, toluenesulfonic, camphorsulfonic, and similarly known acceptable acids. The compounds can also be used in the form of esters, carbamates and other conventional prodrug forms, which when administered in such form, convert to the active moiety in vivo. When using the term compound herein, it is understood that all salts are included.

The present invention accordingly provides a pharmaceutical composition which comprises a compound of this invention in combination or association with a pharmaceutically acceptable carrier. In particular, the present invention provides a pharmaceutical composition which comprises an effective amount of a compound of this invention and a pharmaceutically acceptable carrier.

The term “pharmaceutically acceptable salt” as used herein is intended to include the non-toxic acid addition salts with inorganic or organic acids, e.g. salts with acids such as hydrochloric, phosphoric, sulfuric, maleic, acetic, citric, succinic, benzoic, fumaric, mandelic, p-toluene-sulfonic, methanesulfonic, ascorbic, lactic, gluconic, trifluoroacetic, hydroiodic, hydrobromic, and the like. Examples of pharmaceutically acceptable salts include, but are not limited to, mineral or organic acid salts of basic residues such as amines; alkali or organic salts of acidic residues such as carboxylic acids; and the like.

Pharmaceutically acceptable salts of the compounds of the invention can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, p. 1418, the disclosure of which is hereby incorporated by reference.

B. Methods of Using the Compounds of the Present Invention

As stated above, the compounds of the present invention are useful as chemopreventive and chemotherapeutic agents.

Accordingly, specifically included as methods of the present invention are methods of preventing and/or treating cancer in a mammalian subject, comprising administering an effective amount to the mammalian subject a pharmaceutical preparation comprising a compound of the present invention and a pharmaceutical carrier.

Another method of the present invention is a method of promoting anti-tumor activity in a mammalian subject, comprising administering a effective amount of a compound of the present invention and a pharmaceutical carrier.

Yet another method of the present invention is a method for preventing and/or treating carcinogenesis comprising: administering to a mammalian subject having precancerours precursors, but not having cancer, a pharmaceutical preparation comprising a compound of the present invention and a pharmaceutically acceptable salt in an amount effective to prevent the occurrence of the cancer or precancerous condition or to slow, halt or reverse the progression of the cancer or precancerous condition.

The invention also includes methods of preventing the occurrence or progression of a cancer or a precancerous condition in a mammal comprising administering to the mammal an effective amount of a composition comprising a compound of the present invention and a pharmaceutically acceptable carrier.

The present invention also includes methods of treating or preventing cancer in a mammalian subject, comprising administering an effective amount to the mammalian subject a pharmaceutical preparation comprising a chemopreventive or chemotherapeutic compound of the following formula:

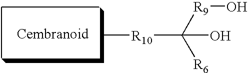

wherein the variables are defined above. Further, R₆ is H or substituted or unsubstituted alkyl (including C₁, C₂, C₃, C₄, C₅, and C₆ alkyl groups), R₉is a bond or a substituted or unsubstituted alkyl group (including C₁, C₂, C₃, C₄, C₅, and C₆ alkyl groups); and R₁₀ is a bond or a substituted or unsubstituted alkyl group (including C₁, C₂, C₃, C₄, C₅, and C₆ alkyl groups). The cembranoid compound is understood as being substituted or unsubstituted. The cembranoid compound may further have the same substituents as listed for the aryl and Het groups, above.

Finally, embodiments of the present invention further include methods of treating and preventing tumors and methods of treating and preventing carcinogenesis. The compounds of the present invention also have anti-carcinogenic promoter and anti-tumor promoter activity. The compounds of the present invention may be used to prevent or treat oxidative stress induced by phorbol ester-type tumor promoters (TPA). As stated in U.S. Pat. No. 5,591,773, incorporated herein by reference, TPA has been found induce oxidative stress that also causes opacity in bovine eye lens. Also see Wei, H. and Frenkel, K. In vivo formation of oxidized bases in tumor promoter-treated mouse skin. Cancer Res. 51: 4443-4449, 1991.; and Wei, H. and Frenkel, K. Suppression of tumor promoter-induced oxidative events and DNA damage in vivo by sarcophytol A: a possible mechanism of antipromotion. Cancer Res. 52: 2298-2303, 1992; both of which are incorporated herein by reference. Additionally, as stated in U.S. Pat. No. 5,591,773, it has been found that agents possessing anti-tumor-promoting properties in vivo, also suppress inflammatory processes. Therefore, the compounds of the present invention may be used to obtain anti-inflammatory activity for use in treating conditions such as, for example, arthritis.

With respect to all methods of the present invention, it is understood that the compounds/compositions are administered in therapeutically effective amounts. The term “therapeutically effective amount” of a compound of this invention means an amount effective to achieve the desired result (e.g., promote anti-tumor activity) in a host.

Cancer, as used herein includes, but is not limited to, malignant tumors, adenocarcinomas, carcinomas, sarcomas, malignant neoplasms, and leukemias. In particular epithelial cell derived cancers are intended to be encompassed by this invention. Examples of epithelial cell derived cancers that may be treated by the methods described herein include, but are not limited to, breast cancer, colon cancer, ovarian cancer, lung cancer or prostate cancer. Such cancers may be caused by, chromosomal abnormalities, degenerative growth and developmental disorders, mitogenic agents, ultraviolet radiating (UV), viral infections, oncogenes, mutations in genes, in-appropriate expression of a gene and presentation on a cell, or carcinogenic agent.

Administration of the compositions of the present invention may be for either a prophylactic or therapeutic use. When provided prophylactically, a compound of the present invention is provided in advance of any symptoms due to the cancer afflicting the individual. Additionally, a compound of the present invention may be administered during or after cancer treatment to help prevent the reoccurrence of cancer. The prophylactic administration of the composition is intended as a chemopreventive therapy and serves to either prevent initiation of malignant cells or arrest or reverse the progression of transformed premalignant cells to malignant disease.

When provided therapeutically the composition is provided at or after the onset of the disease. The therapeutic administration of the composition of this invention serves to attenuate or alleviate the cancer or facilitate regression of the cancer afflicting the individual. The term individual is intended to include any animal, preferably a mammal, and most preferably a human. Veterinary uses are intended to be encompassed by this definition.

In one embodiment of this invention, individuals at high risk for a particular cancer, or at high risk of reoccurrence of a cancer or who have known risk factors are prophylactically treated with the methods and compositions described herein. By way of example, such individuals may include those with a familial history for either early or late onset of cancer, and individuals who are being or have been treated for a cancer or cancer-related illness.

General ranges of suitable effective prophylactic dosages that may be used are about 0.1 mg/kg of body weight per day to about 500 mg/kg/day, a further range is about 0.5 mg/kg/day to about 250 mg/kg/day, and as a further range, about 1 mg/kg/day to about 100 mg/kg/day.

The daily dose of the compound may be administered in a single dose or in portions at various hours of the day. Initially, a higher dosage may be required and may be reduced over time when the optimal initial response is obtained. By way of example, treatment may be continuous for days, weeks, or years, or may be at intervals with intervening rest periods. The dosage may be modified in accordance with other treatments the individual may be receiving. One of skill in the art will appreciate that individualization of dosage may be required to achieve the maximum effect for a given individual. It is further understood by one skilled in the art that the dosage administered to a individual being treated may vary depending on the individuals age, severity or stage of the disease and response to the course of treatment. One skilled in the art will know the clinical parameters to evaluate to determine proper dosage for the individual being treated by the methods described herein.

Additional pharmaceutical methods may be employed to control the duration of action. Controlled release preparations may be achieved through the use of polymer to complex or absorb the proteins or their derivatives. The controlled delivery may be exercised by selecting appropriate macromolecules (for example polyester, polyamino acids, polyvinyl, pyrrolidone, ethylenevinylacetate, methylcellulose, carboxymethylcellulose, or protamine sulfate) and the concentration of macromolecules as well as the methods of incorporation in order to control release. Alternatively, instead of incorporating these agents into polymeric particles, it is possible to entrap these materials in microcapsules prepared, for example, by coacervation techniques or by interfacial polymerization.

When oral preparations are desired, the component may be combined with typical carriers/excipients, such as lactose, sucrose, starch, talc magnesium stearate, crystalline cellulose, methyl cellulose, carboxymethyl cellulose, glycerin, sodium alginate or gum arabic among others. The only limitation with resect to the carrier is that it does not deleteriously react with the active compound or is not deleterious to the recipient thereof.

The pharmaceutical preparations can be sterilized and if desired mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously react with the active compounds.

As stated above, the administration of the compositions or of each individual component of the present invention may be for either a prophylactic or therapeutic purpose. The methods and compositions used herein may be used alone in prophylactic or therapeutic uses or in conjunction with additional therapies known to those skilled in the art in the prevention or treatment of cancer. Alternatively the methods and compositions described herein may be used as adjunct therapy.

It will be appreciated that the actual preferred amounts of active compounds used in a given therapy will vary according to the specific compound being utilized, the particular compositions formulated, the mode of application, the particular site of administration, etc. Optimal administration rates for a given protocol of administration can be readily ascertained by those skilled in the art using conventional dosage determination tests.

B. Experimental Description/Examples/Preparation of Compounds of the Present Invention

This section is presented as the best mode and for exemplary purposes. Specifically, the information and examples provided herein are intended to demonstrate certain embodiments of the present invention and not to be construed as limiting the scope of the present invention.

Sarcophine (3) was treated with selenium dioxide in dioxane at different temperatures. At room temperature it underwent a selective oxidation to yield product 4 along with product 5, while in refluxing dioxane the reaction gave two products, 6 and 7.

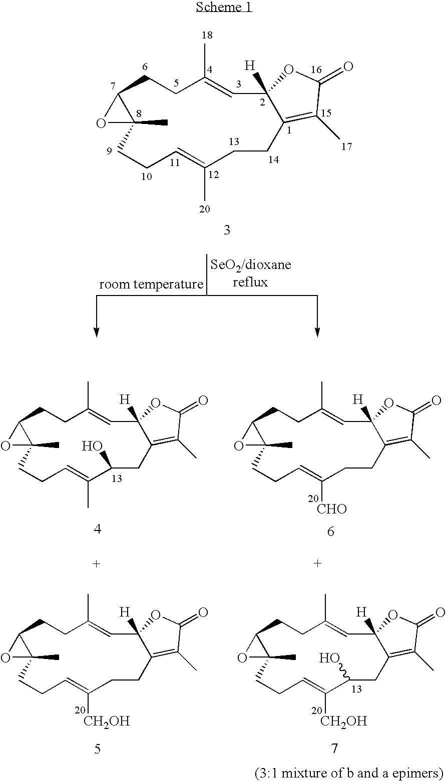

Compound 4, obtained at room temperature was identified as 13S-hydroxysarcophine. Compound 5 was identified as 20-hydroxysarcophine. Compound 6 was identified as sarcophine-20-carboxyaldehyde. Compound 7 was identified as a mixture of 13S,20- and 13R,20-dihydroxysarcophine, respectively.

Without being bound by theory, the most likely mechanism for the allylic hydroxylation of sarcophine (3) with selenium dioxide involves addition of SeO₂ to olefinic bond in 3 to form selenoxide intermediates 8, which undergo [2, 3] sigmatropic rearrangement to yield hydroxysarcophine precursors 9 (Scheme 2).

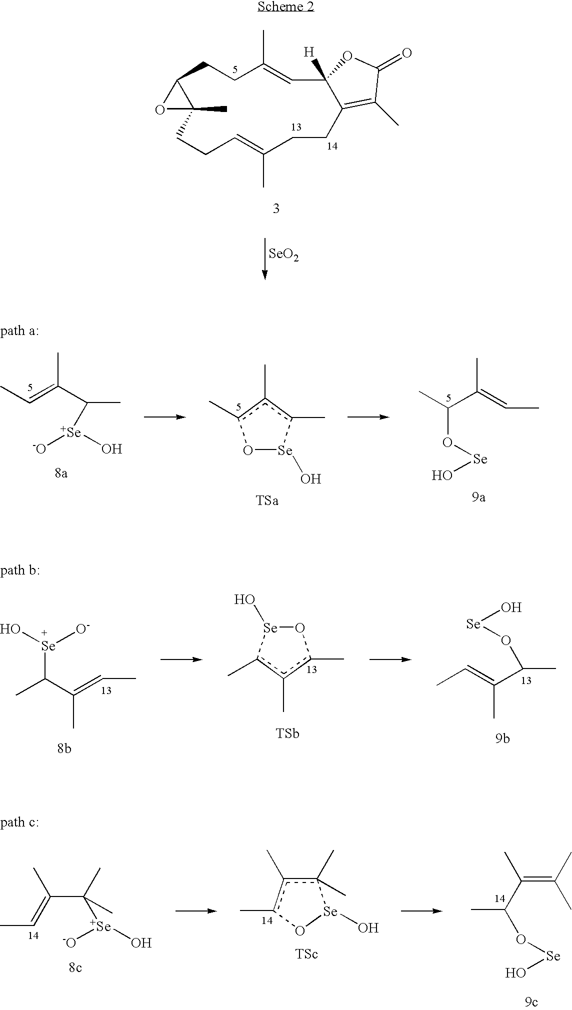

According to Guillemonat rules, three positions (C-5, C-13, and C-14) should be preferentially hydroxylated. At room temperature, however, no reaction at C-5 and C-14 was observed and the C-13 position was only affected. In order to explain this regioselectivity, the reaction coordinates of the [2, 3]-sigmatropic rearrangement of 8 were calculated using density functional method.

The coordinates indicate that the process involving formation of 9b from 8b via TSb has the lowest activation energy. The lower stability of Tsa and TSc may be ascribed to electronic repulsion between the selenoxide moiety and the oxygen of the lactone ring. Consequently, 13-hydroxysarcophine may be formed at room temperature.

Without being bound by theory, the stereoselective β-hydroxylation at the C-13 can be explained on the basis of conformational properties of sarcophine (3). Conformational analysis of 3 revealed the presence of several kinds of conformation. Only three types of conformation, however, seem to be accountable for the reaction at room temperature because the C2-C11 segment in 3 can be assumed to be less flexible on the basis of the restricted freedom of the macrocycle arising from a half chair conformation for C7-C11 segment and a large dihedral angle between H-2β and H-3. The local minimum conformers of these three conformations correspond to conformers A, B, and C, where C20 methyl group is directed away from C18 methyl group, the C20 is directed toward C-18, and where the C20 is directed opposite to C-18, respectively.

The conformer with much higher HOMO energy may be the most reactive for the electrophilic attack. In this case, however, HOMO energies may be similar among these three conformers. Therefore, the lowest-energy conformer A may be the most susceptible for the electrophilic addition of selenium dioxide. In the conformer A, the α face of the C11-C12 double bond is hindered, forcing the addition of selenium dioxide from the same direction as C-18 methyl group in relation to the macrocycle.

At reflux temperature, the addition of selenium dioxide to the C11-C12 double bond occurred again. In this case, hydroxylation of C-13 position is less stereoselective and it is followed by further hydroxylation on carbon C-20 forming the mixture of diols 7. The 20-hydroxysarcophine formed at room temperature is also oxidized further to the corresponding aldehyde 6.

Reaction of the lactone ring-opened sarcophine analog (10) with selenium dioxide, provided compounds of the present invention, including an epoxycembranediol (11) and derivatives (12-15) (Scheme 3).

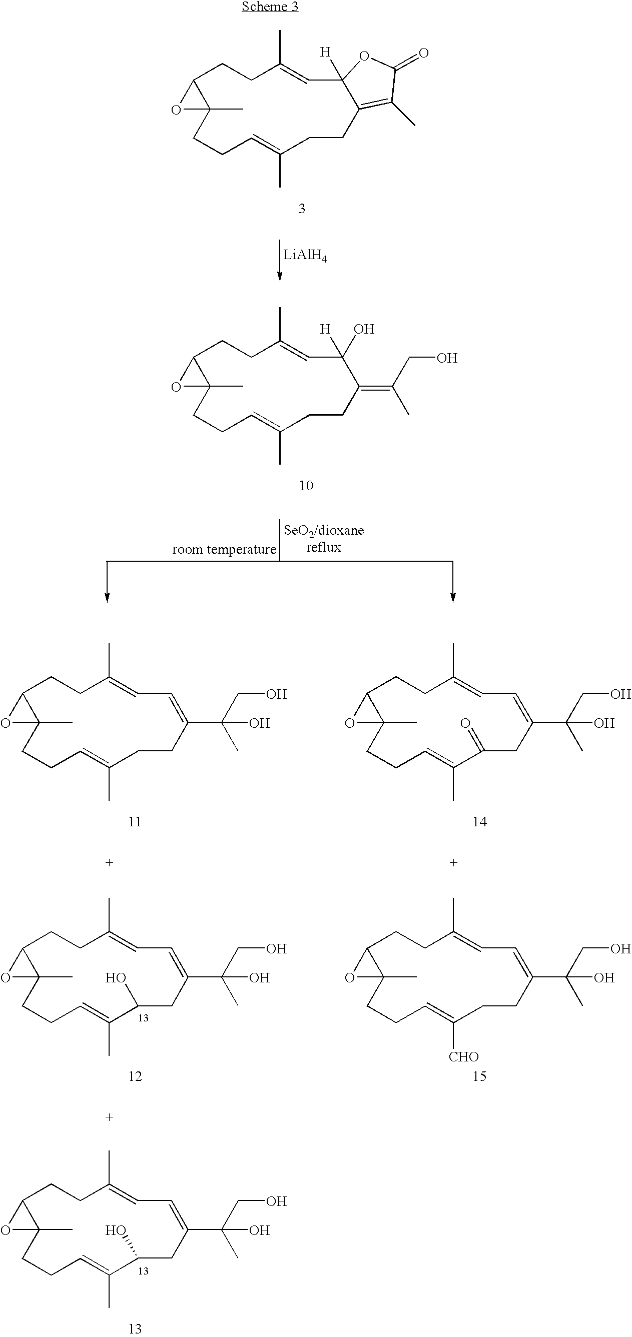

At room temperature this example provided compounds of the present invention 11, 12, and 13, and while in refluxing dioxane provided compounds of the present invention 14 and 15.

Compound 11 was shown to have molecular formula C₂₀H₃₂O₃by HRMS. The ¹H-NMR spectrum showed no signal corresponding to proton H-2β present in the starting material 10. Instead, additional signal of vinylic proton at δ 6.43 (d, J=11.3 Hz) coupled with vinylic proton H-3 (δ 5.96, d, J=11.3 Hz) appears in the spectrum, suggesting the presence of conjugated double bonds system. The UV spectrum (λₘₐₓ 252 nm, ε7510) supports the presence of s-trans-1,1,4,4-tetrasubstituted diene system. In ¹³C NMR spectrum a new quaternary signal appeared at δ 76.6. All of these suggest that compound 11 may be a product of allylic rearrangement of 10 in the presence of selenious acid. Two possible directions of such rearrangement should be considered, forming tertiary alcohol 11a or 11b, also compounds of the present invention.

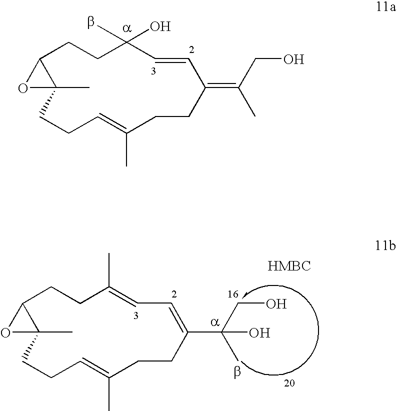

Compound 11 was identified as 7,8-epoxy-1,3,11-cembratriene-15α,16-diol.

The compounds 12 and 13 of the present invention had the molecular formula C₂₀H₃₂O₄, suggesting that they are hydroxylated derivatives of 11. The signal patterns in the ¹H and ¹³C NMR spectra of 12 were found to be identical with those of 13. The HMBC experiment indicated that both compounds have hydroxyl groups at C-13 position because α-protons adjacent to hydroxyl groups (δ 4.27, 3.99) showed correlations with methyl carbons C-20 (δ 10.7, 11.6), respectively. They were identified as 13S(12) and 13R(13) isomers of 7,8-epoxy-1,3,11-cembratriene-13,15α,16triol. The structure of the compound 13 was independently confirmed by X-ray crystallography.

By comparison with absolute configuration of sarcophine it was possible to determine the configuration of C-13 and C-15 hydroxyl groups as R. With the help of X-ray crystallography we were able to establish the configuration of C-15 hydroxyl group in the 1,2-propandiol moiety, otherwise difficult to determine by NMR spectroscopy due to the free rotation around C-1 C-15 bond.

The R configuration of the stereogenic center at C-15 suggests that the allylic rearrangement of compound 10 to 11 may have occurred in the concerted S_{N}i′ process from the α-side of the molecule.

Compound 14 had the molecular formula C₂₀H₃₀O₄ and the characteristic infrared band at 1650 cm⁻¹. The ¹³C NMR spectrum showed the signal of a carbonyl group (δ 204.1) that correlated in HMBC spectrum with methylene protons H-14 (δ 2.98, 4.06) and the protons of the C-20 methyl group. This confirms the structure of compound 14 as 7,8-epoxy-13-oxo-1,3,11-cembratriene-15α,16-diol.

HRMS spectrum for the compound 15 indicated the molecular formula C₂₀H₃₀O₄. IR (1681 cm⁻¹), ¹H and ¹³C NMR spectra (δ 9.37, 195.1) suggested the presence of an aldehyde group. The absence of ¹H methyl signal at δ1.64-1.72, which existed in compounds 11-14, suggested that C-20 methyl group was converted into aldehyde group, and the product 15 has the structure of 7,8-epoxy-1,3,11-cembratriene-15α,16-triol-20-carboxyaldehyde.

When reduced sarcophine (10) was treated with equimolar amounts of selenium dioxide in dioxane at room temperature, 15α,16-diol (11) was obtained without the formation of 13,15,16-triols (12) and (13). This fact suggests that the conversion of 10 into 11 is stoichiometric reaction with SeO₂ and that the hydroxylation of the later with an excess of SeO₂ provides 12 and 13.

Similarly to what we described earlier, three positions, C-5, C-13, and C-14 in 10 should be preferentially hydroxylated. At room temperature, 13-hydroxy derivatives 12 and 13 are predominantly formed, while C-5 and C-14 are not affected. The ketone 14 and aldehyde 15 may be formed by further oxidation of triols 12 and 13, and diol 11 respectively, at reflux temperature.

After reduction of the unsaturated lactone ring, hydroxylation is no more stereoselective, and the equal quantities of 13S-(12) and 13R-(13) isomers of 7,8-epoxy-1,3,11-cembratriene-13,15α,16-triol are formed.

In 1998 ElSayed, Hamann and others published the work on microbial transformations of sarcophine that included hydroxylation of the molecule. See Sayed et al., J. Org. Chem. 1998, 63, 7449-7455. An average of four compounds were produced in each microbial reactions with yields ranging from 0.5-5%. The hydroxylation took place at positions 4, 5, 7, 8, 9, 10 and 19.

The present inventors have determined that modifications of sarcophine affect different sites of the molecule, and may serve as useful complementations of different methods in drug development from natural products.

### EXAMPLE 1

### Reaction of Reduced Sarcophine (10) with Selenium Dioxide at Room Temperature

Selenium dioxide (35.5 mg, 0.32 mmol) was added to a solution of reduced sarcophine 10 (50 mg, 0.16 mmol) in dry 1,4-dioxane (15 mL), and the reaction mixture was stirred at room temperature for 4 hours. Water was then added, and the product was extracted with CH₂Cl₂. The CH₂Cl₂ layer was washed with saturated NaHCO₃ solution and dried over anhydrous Na₂SO₄. The solvent was evaporated and the residue was chromatographed on silica gel using hexane-acetone (1:1) as an eluent to obtain 15,16-diol 11 (7 mg, 13%) and 13β,15,16-triol 12 (12 mg, 23% and 13α,15,16-triol 13 (11 mg, 21%). They were re-purified by preparative TLC plate (silica gel, 1:2 hexane-EtOAc for 11, 1:5 hexane-EtOAc for 12 and 13).

7,8-Epoxy-1,3,11-cembratriene-15R(α),16-diol (11): colorless oil; UV (MeOH) λₘₐₓ (log ε) 252 (7510) nm; IR (neat) νₘₐₓ 3406 (OH) cm⁻¹; ¹H NMR (CDCl₃) δ 6.43 (1H, d, J=11.3 Hz, H-2), 5.96 (1H, d, J=11.3 Hz, H-3), 5.10-5.13 (1H, m, H-11), 3.66 (1H, d, J=11.0 Hz, H-16), 3.46 (1H, d, J=11.0 Hz, H-16), 2.84 (1H, t, J=4.8 Hz, H-7), 2.04-2.34 (12H, m, H₂-5, 6, 9, 10, 13, 14), 1.80 (3H, s, H-18), 1.64 (3H, s, H-20), 1.33 (3H, s, H-17), 1.27 (3H, s, H-19); ¹³C NMR (CDCl₃) δ 144.4 (s, C-1), 138.3 (s, C-4), 136.0 (s, C-12), 125.7 (d, C-11), 121.4 (d, C-2), 120.5, (d, C-3), 76.6 (s, C-15), 69.3 (t, C-16), 62.7 (d, C-7), 60.4 (s, C-8), 41.6 (t, C-13), 38.9 (t, C-9), 36.0 (t, C-5), 26.7 (t, C-14), 26.3 (t, C-6), 24.7 (q, C-17), 23.6 (t, C-10), 18.2 (q, C-18), 17.4 (q, C-19),16.5 (q, C-20); HRESMS m/z 343.2237 (calcd for C₂₀H₃₂O₃Na (M+Na)⁺343.2249).

7,8-Epoxy-1,3,11-cembratriene-13S(β),15R(α),16-triol (12): colorless oil; [α]_{D}³⁰=−37.0° (c 0.3, CH₂Cl₂); IR (KBr) νₘₐₓ 3422 (OH) cm⁻¹; ¹H NMR (CDCl₃) δ 6.41 (1H, d, J=9.5 Hz, H-2), 5.89 (1H, d, J=9.5 Hz, H-3), 5.46-5.49 (1H, m, H-11), 4.27 (1H, dd, J=7.3, 2.1 Hz, H-13), 3.76 (1H, d, J=11.1 Hz, H-16), 3.42 (1H, d, J=11.1 Hz, H-16), 2.91 (1H, dd, J=7.5, 3.2 Hz, H-7), 2.40 (1H, dd, J=14.3, 7.3 Hz, H-14), 2.17-2.52 (9H, m, H₂-5, 6, 9, 10, and H-14), 1.77 (3H, s, H-18), 1.66 (3H, s, H-20), 1.33 (3H, s, H-17), 1.30 (3H, s, H-19); ¹³C NMR (CDCl₃) δ 142.9 (s, C-1), 139.8 (s, C-4), 139.1 (s, C-12), 126.3 (d, C-11), 124.0 (d, C-2) 120.0 (d, C-3), 80.8 (d, C-13), 76.2 (s, C-15), 69.6 (t, C-16), 62.7 (d, C-7), 60.5 (s, C-8), 38.6 (t, C-9), 35.5 (t, C-14), 34.8 (t, C-5), 26.9 (t, C-6), 25.3 (q, C-17), 23.0 (t, C-10), 18.8 (q, C-18), 17.2 (q, C-19), 10.7 (q, C-20); HRESMS m/z 359.2245 (calcd for C₂₀H₃₂O₄Na (M+Na)⁺359.2198).

7,8-Epoxy-1,3,11-cembratriene-13R(α),15R(α),16-triol (13): mp 141-143° C.; [α]_{D}²³=+1.5° (c 0.2, CH₂Cl₂); IR (KBr) νₘₐₓ 3387 (OH) cm⁻¹; ¹H NMR (CDCl₃) δ 6.28 (1H, d, J=11.0 Hz, H-2), 5.80 (1H, d, J=11.0 Hz, H-3), 5.26-5.29 (1H, m, H-11), 3.99 (1H, dd, J=6.0, 2.2 Hz, H-13), 3.78 (1H, d, J=11.0 Hz, H-16), 3.61 (1H, d, J=11.0 Hz, H-16), 2.72 (1H, dd, J=5.9, 3.4 Hz, H-7), 2.50 (1H, dd, J=14.4, 6.0 Hz, H-14), 2.01-2.33 (9H, m, H₂-5, 6, 9, 10, and H-14), 1.80 (3H, s, H-18), 1.72 (3H, s, H-20), 1.34 (3H, s, H-17), 1.25 (s, 3H, H-19); ¹³C NMR (CDCl₃) δ 143.1 (s, C-1), 138.8 (s, C-4), 138.2 (s, C-12), 128.2 (d, C-11), 121.5 (d, C-2) 119.8 (d, C-3), 81.8 (d, C-13), 75.9 (s, C-15), 69.7 (t, C-16), 62.9 (d, C-7), 60.5 (s, C-8), 38.9 (t, C-9), 36.1 (t, C-5), 34.6 (t, C-14), 26.0 (q, C-17), 25.8 (t, C-6), 23.6 (t, C-10), 18.6 (q, C-18), 17.3 (q, C-19), 11.6 (q, C-20); HRESMS m/z 359.2224 (calcd for C₂₀H₃₂O₄Na (M+Na)⁺359.2198).

### EXAMPLE 2

### Reaction of Reduced Sarcophine (10) with Selenium Dioxide at 100° C.

Selenium dioxide (67 mg, 0.6 mmol) was added to a solution of reduced sarcophine 10 (96 mg, 0.3 mmol) in dry 1,4-dioxane (30 mL), and the reaction mixture was stirred at reflux temperature for 1 hour. The products were worked-up as described for the previously to yield the ketone 14 (17 mg, 17%) and the aldehyde 15 (12 mg, 12%). They were also re-purified by preparative TLC plate (silica gel, 1:1 CH₂Cl₂-EtOAc).

7,8-Epoxy-13-oxo-1,3,11-cembratriene-15R(α),16-diol (14): colorless oil, [α]_{D}³⁰=−13.3° (c 0.3, CH₂Cl₂); IR (neat) νₘₐₓ 3414 (OH), 1650 (C═O) cm⁻¹; ¹H NMR (CDCl₃) δ 6.90-6.93 (1H, m, H-11), 6.60 (1H, d, J=10.6 Hz, H-2), 5.51 (1H, d, J=10.6 Hz, H-3), 4.06 (1H, d, J=15.6 Hz, H-14), 3.79 (1H, d, J=12.0 Hz, H-16), 3.39 (1H, d, J=12.0 Hz, H-16),2.98 (1H, d, J=15.6 Hz, H-14), 2.79 (1H, d, J=7.2 Hz, H-7), 2.18-2.60 (8H, m, H₂-5, 6, 9, 10), 1.88 (3H, s, H-20), 1.79 (3H, s, H-18), 1.29 (3H, s, H-19), 1.23 (3H, s, H-17); ¹³C NMR (CDCl₃) δ 204.1 (s, C-13), 145.4 (d, C-11), 140.8 (s, C-1), 139.4, (s, C-12), 138.4 (s, C-4), 123.4 (d, C-2), 118.4, (d, C-3), 76.4 (s, C-15), 68.8 (t, C-16), 62.5 (d, C-7), 59.4 (s, C-8), 38.5 (t, C-9), 35.1 (t, C-14), 34.9 (t, C-5), 26.3 (t, C-6), 23.7 (q, C-17), 23.6 (t, C-10) 19.0 (q, C-18), 16.8 (q, C-19), 11.8 (q, C-20); HRESMS m/z 357.1997 (caled for C₂₀H₃₀O₄Na (M+Na)⁺357.2042).

7,8-Epoxy-1,3,11-cembratriene-15R(α),16-diol-20-carboxyaldehyde (15): colorless oil, [α]_{D}³⁰=−49.3° (c 0.3, CH₂Cl₂); IR (neat) νₘₐₓ 3415 (OH), 1681 (C═O) cm⁻¹; ¹H NMR (CDCl₃) δ 9.37 (1H, s, H-20), 6.66 (1H, dd, J=9.9, 6.5 Hz, H-11), 6.49 (1H, d, J=10.6 Hz, H-2), 6.12 (1H, d, J=10.6 Hz, H-3), 3.64 (1H, d, J=11.1 Hz, H-16), 3.44 (1H, d, J=11.1 Hz, H-16), 2.76 (1H, dd, J=10.4, 2.7 Hz, H-7), 2.06-2.63 (12H, m, H₂-5, 6, 9, 10, 13, 14), 1.85 (3H, s, H-18), 1.43 (3H, s, H-17), 1.34 (3H, s, H-19); ¹³C NMR (CDCl₃) δ 195.1 (d, C-20), 153.0 (d, C-11), 143.6 (s, C-1), 142.7 (s, C-12), 137.7 (s, C-4), 123.1 (d, C-3), 121.8 (d, C-2), 76.5 (s, C-15), 68.9 (t, C-16), 63.0 (d, C-7), 61.7 (s, C-8), 39.5 (t, C-9), 38.0 (t, C-5), 27.7 (t, C-13), 27.2 (t, C-14), 25.7 (t, C-6), 25.4 (q, C-17), 23.1 (t, C-10), 16.6 (q, C-19), 14.5 (q, C-18); HRESMS m/z 357.2013 (calcd for C₂₀H₃₀O₄Na (M+Na)⁺357.2042).

### EXAMPLE 3

### Inhibition of EBV-EA Activation

The inhibition of EBV-EA activation was assayed using Raji cells (virus non-producer type). The indicator cells (Raji, 1×10⁶/ml) were incubated at 37° C. for 48 h in 1 ml of medium containing n-butyric acid (4 mmol), TPA(12-O-tetradecanoylphorbol 13-acetate)(32 pmol=20 ng in 2 μl DMSO) as inducer and various amounts of test compounds in 5 μl DMSO. Smears were made from the cell suspension, and the activated cells, which were stained by EBV-EA positive serum from nasopharygeal carcinoma patients were detected by an indirect immunofluorescence technique. For each compound, assays were performed in triplicate. The average EBV-EA induction of the test compounds was expressed as a relative ratio to the control experiment (100%), which was carried out only with n-butyric acid plus TPA. The viability of treated Raji cells was assayed by the Trypan Blue staining method.

Results of this assay (Table 1) have shown that compounds of the present invention are effective chemopreventive and chemotherapeutic agents.

TABLE 1

| Concentration^{a} | x 1000 | x 500 | x 100 | x 10 |
|---|---|---|---|---|
| 3 | 19.6^{b}(60^{c}) | 67.5(>80) | 84.0(>80) | 100(>80) |
| 4 | 14.2(60) | 62.8(>80) | 81.3(>80) | 100(>80) |
| 6 | 12.7(60) | 60.8(>80) | 78.2(>80) | 96.0(>80) |
| 10 | 9.6(60) | 57.3(>80) | 75.5(>80) | 94.1(>80) |
| 12 | 5.9(60) | 55.8(>80) | 74.6(>80) | 93.8(>80) |
| 13 | 5.7(60) | 54.2(>80) | 72.9(>80) | 92.6(>80) |
| 14 | 8.3(60) | 56.7(>80) | 75.1(>80) | 94.0(>80) |
| 15 | 3.6(60) | 52.4(>80) | 71.3(>80) | 90.6(>80) |
| Sarcophytol-A | 11.7(50) | 60.4(>80) | 79.1(>80) | 97.3(>80) |

^{a}Concentrations are expressed in mol ratio/TPA. TPA was used in 32 pmol concentration, therefore, ex. x 1000 means used sample in 32 nmol/mL.
^{b}Values represent percentages relative to the positive control value (100%)
^{c}Values in parentheses represent viability percentages of Raji cells (indicator cells).

### EXAMPLE 4

### In Vivo Chemopreventive Activity

In this Example, four compounds of the present invention (9-12) were evaluated for their in vivo chemopreventive activity using the two-stage mouse skin carcinogenesis model.

In the two-stage mouse skin carcinogenesis model induced by DMBA/TPA, the animals (specific pathogen-free female ICR, 6 weeks old) were divided into 5 groups, 15 mice each. The back of each mouse was shaved with surgical clippers, and the mice were topically treated with DMBA (100 μg, 390 nmol) in acetone (0.1 ml) as an initiation treatment. For the positive control group, 1 week after initiation with DMBA, mice were promoted by the application with TPA (1 μg, 1.7 nmol) in acetone (0.1 ml) twice a week. The other four groups received a topical application of each of the sarcophine semi-synthetic derivatives (85 nm) 1 hour before application of the tumor promoter. The incidence of papillomas was observed weekly for 20 weeks. The percentage of mice bearing papillomas and the average number of papillomas per mouse were recorded.

In the positive control group, mice showed 100% incidence of papillomas within 10 weeks. Mice which were treated with the sarcophine derivatives showed 26.6% incidence of papillomas after the same period (10 weeks). They showed 53.3-60% and 80-86.6% papillomas formation after 15 weeks and 20 weeks, respectively.

The tumor inhibitory effects were also seen as a reduction in the multiplicity of papillomas. In the positive control group, the number of papillomas per mice was 5.4, 7.8 and 9.3 after 10 weeks, 15 weeks, and 20 weeks, respectively. In the groups treated with the sarcophine derivatives, the number of papillomas per mouse was 1.1-1.6, 2.7-3.1 and 4.6-5.1 after 10 weeks, 15 weeks and 20 weeks, respectively.

The reduction of both criteria (% incidence of papillomas and multiplicity of papillomas) in those after administration of the four compounds of the present invention (compounds 9-12) compared to the positive control group, indicated chemopreventive activity.

TABLE 2

| | control | | | | | | | | | |
|---|---|---|---|---|---|---|---|---|---|---|
| Weeks | Papilloma | | 9 | | 10 | | 11 | | 12 | |
| of pro- | Bearer | Papillomas/ | Papilloma | Papillomas/ | Papilloma | Papillomas/ | Papilloma | Papillomas/ | Papilloma | Papillomas/ |
| motion | (%) | mouse | Bearer (%) | mouse | Bearer (%) | mouse | Bearer (%) | mouse | Bearer (%) | mouse |
| 1 | 0.0 | 0.0 | 0.0 | 0.0 | 0.0 | 0.0 | 0.0 | 0.0 | 0.0 | 0.0 |
| 2 | 0.0 | 0.0 | 0.0 | 0.0 | 0.0 | 0.0 | 0.0 | 0.0 | 0.0 | 0.0 |
| 3 | 0.0 | 0.0 | 0.0 | 0.0 | 0.0 | 0.0 | 0.0 | 0.0 | 0.0 | 0.0 |
| 4 | 0.0 | 0.0 | 0.0 | 0.0 | 0.0 | 0.0 | 0.0 | 0.0 | 0.0 | 0.0 |
| 5 | 0.0 | 0.0 | 0.0 | 0.0 | 0.0 | 0.0 | 0.0 | 0.0 | 0.0 | 0.0 |
| 6 | 20.0 | 1.0 | 0.0 | 0.0 | 0.0 | 0.0 | 0.0 | 0.0 | 0.0 | 0.0 |
| 7 | 33.3 | 2.0 | 0.0 | 0.0 | 0.0 | 0.0 | 0.0 | 0.0 | 0.0 | 0.0 |
| 8 | 60.0 | 3.3 | 13.3 | 0.5 | 13.3 | 0.5 | 13.3 | 0.6 | 13.3 | 0.2 |
| 9 | 90.0 | 4.7 | 20.0 | 1.1 | 13.3 | 0.9 | 20.0 | 1.3 | 20.0 | 0.5 |
| 10* | 100 | 5.4 | 26.6 | 1.6 | 26.6 | 1.3 | 26.6 | 1.6 | 26.6 | 1.1 |
| 11 | 100 | 5.9 | 33.3 | 1.8 | 33.3 | 1.8 | 40.0 | 1.9 | 33.3 | 1.5 |
| 12 | 100 | 6.3 | 40.0 | 2.0 | 40.0 | 2.1 | 46.6 | 2.1 | 33.3 | 2.0 |
| 13 | 100 | 6.8 | 46.6 | 2.3 | 46.6 | 2.3 | 53.3 | 2.4 | 40.0 | 2.2 |
| 14 | 100 | 7.4 | 53.3 | 2.6 | 53.3 | 2.5 | 53.3 | 2.7 | 46.6 | 2.7 |
| 15* | 100 | 7.8 | 60.0 | 2.9 | 60.0 | 2.7 | 60.0 | 3.0 | 53.3 | 3.1 |
| 16 | 100 | 8.3 | 73.3 | 3.3 | 73.3 | 3.1 | 73.3 | 3.4 | 60.0 | 3.7 |
| 17 | 100 | 8.6 | 80.0 | 3.6 | 73.3 | 3.6 | 80.0 | 3.7 | 66.6 | 4.0 |
| 18 | 100 | 8.7 | 80.0 | 4.3 | 80.0 | 4.0 | 80.0 | 4.2 | 73.3 | 4.1 |
| 19 | 100 | 9.0 | 80.0 | 4.7 | 80.0 | 4.6 | 86.6 | 4.7 | 73.3 | 4.4 |
| 20* | 100 | 9.3 | 80.0 | 5.0 | 80.0 | 4.8 | 86.6 | 5.1 | 80.0 | 4.6 |

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the Specification and Example be considered as exemplary only, and not intended to limit the scope and spirit of the invention.

Unless otherwise indicated, all numbers expressing quantities of ingredients, properties such as reaction conditions, and so forth used in the Specification and Claims are to be understood as being modified in all instances by the term “about.” Accordingly, unless indicated to the contrary, the numerical parameters set forth in the Specification and Claims are approximations that may vary depending upon the desired properties sought to be determined by the present invention.

Notwithstanding that the numerical ranges and parameters setting forth the broad scope of the invention are approximations, the numerical values set forth in the experimental or example sections are reported as precisely as possible. Any numerical value, however, inherently contain certain errors necessarily resulting from the standard deviation found in their respective testing measurements.

Throughout this application, various publications are referenced. All such references are incorporated herein by reference.

1. A compound of the following formula, which includes salts thereof: 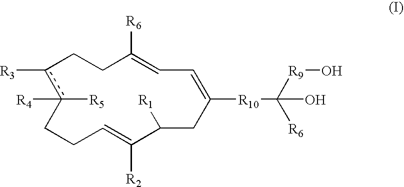
wherein:
R₁ is hydrogen, substituted or unsubstituted alkyl, hydroxyl, acyloxyl, or ketone;
R₂ is hydrogen, substituted or unsubstituted alkyl, hydroxymethyl, carboxaldehyde, or a carboxylic group;
R₃ is hydroxyl, alkoxyl, acyloxyl, amino, alkylamino, arylamino, arylalkylamino, Het-alkylamino, thio, alkylthio, arylthio, arylalkylthio, Het-alkylthio, or together with R₄ forms a double bond or a ring;
R₄ is hydroxyl, alkoxyl, acyloxyl, amino, alkylamino, arylamino, arylalkylamino, Het-alkylamino, thio, alkylthio, arylthio, arylalkylthio, Het-alkylthio, together with R₅ forms a double bond, or together with R₃ forms a double bond or a ring;
R₅ is hydrogen, substituted or unsubstituted alkyl group; or together with R₄ forms a double bond;
R₆ is H or substituted or unsubstituted alkyl group;
R₉ is a bond or a substituted or unsubstituted alkyl group; and
R₁₀ is a bond or a substituted or unsubstituted alkyl group;
with the proviso that when R₃ and R₄ form a double bond, R₂, R₅, R₆, and R₉ are not all methyl and at the same time R₁₀ is not a bond.

2. The compound of claim 1, wherein
R₁ is hydrogen; R₂ is a methyl group; R₃ is a substituted or unsubstituted alkyl group, hydroxyl or methoxyl; and R₄ is a substituted or unsubstituted alkyl group, hydroxyl or methoxyl.

3. The compound of claim 1, wherein:
R₁ is hydrogen; R₂ is a methyl group; and R₃ and R₄ form an oxirane ring.

4. The compound of claim 1, wherein at least one of the alkyl portions of the R₃ substituent is a straight chain or branched alkyl having 1 to 4 carbon atoms.

5. The compound of claim 1, wherein at least one of the aryl portions of the R₃ substituent is phenyl, or naphthyl.

6. The compound of claim 1, wherein at least one of the Het portions of the R₃ substituent is is furyl, pyrrolyl, thienyl, imidazolyl, pyridinyl, pyridazinyl or pyrimidinyl.

7. The compound of claim 1, wherein at least one of the alkyl portions of the R₄ substituent is a straight chain or branched alkyl having 1 to 4 carbon atoms.

8. The compound of claim 1, wherein at least one of the aryl portions of the R₄ substituent is phenyl, or naphthyl.

9. The compound of claim 1, wherein at least one of the Het portions of the R₄ substituent is is furyl, pyrrolyl, thienyl, imidazolyl, pyridinyl, pyridazinyl or pyrimidinyl.

10. The compound of claim 1, wherein R₃ and R₄ form a Het or aryl group.

11. The compound of claim 1, wherein R₃ and R₄ form an oxirane ring, thiirane ring, or a aziridine ring.

12. The compound of claim 1, of the following formula: 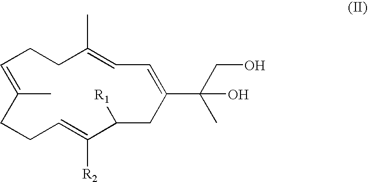
wherein:
R₁ is hydrogen, substituted or unsubstituted alkyl, hydroxyl, acyloxyl, or ketone; and
R₂ is substituted or unsubstituted alkyl, hydroxymethyl, carboxaldehyde, or a carboxylic group;
with the proviso that R₁ is not hydrogen when R₂ is methyl.

13. The compound of claim 1, wherein:
R₆ is H or substituted or unsubstituted alkyl; R₉ is methyl; and R₁₀ is a bond.

14. The compound of claim 1, wherein:
R₁ is hydrogen; R₂ is a methyl group; R₃ is hydroxyl or methoxyl, or together with R₄ forms an oxirane, thiirane, or aziridine ring; R₄ is hydroxyl or methoxyl, or together with R₃ forms an oxirane or aziridine ring; R₅ is methyl; R₆ is H or substituted or unsubstituted alkyl; R₉ is methyl; and R₁₀ is a bond.

15. The compound of claim 1, of the following formula: 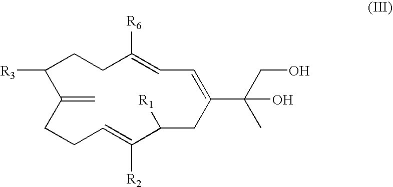
wherein:
R₁ is hydrogen, substituted or unsubstituted alkyl, hydroxyl, acyloxyl, or ketone;
R₂ is hydrogen, substituted or unsubstituted alkyl, hydroxymethyl, carboxaldehyde, or a carboxylic group;
R₃ is hydroxyl, alkoxyl, acyloxyl, amino, alkylamino-, arylamino-, arylalkylamino, Het-alkylamino, thio, alkylthio, arylthio, arylalkylthio, or Het-arylalkylthio; and
R₆ is H or substituted or unsubstituted alkyl.

16. The compound of claim 15, wherein R₁ is hydrogen, R₂ is a methyl group, R₃ is hydroxyl or methoxyl, and R₆ is a methyl group.

17. The compound of claim 1, of the following formula:

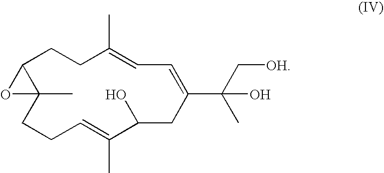

18. The compound of claim 1, of the following formula:

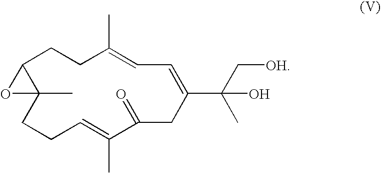

19. The compound of claim 1, of the following formula:

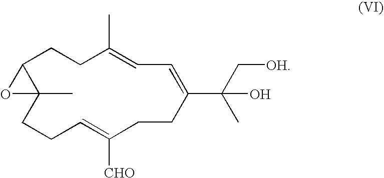

20. A chemopreventive or chemotherapeutic composition, comprising a compound of claim 1 and a pharmaceutically acceptable carrier.

21. A method of preventing cancer in a mammalian subject, comprising administering an effective amount to the mammalian subject a pharmaceutical preparation comprising a compound of claim 1 and a pharmaceutical carrier.

22. A method of treating cancer in a mammalian subject, comprising administering an effective amount to the mammalian subject a pharmaceutical preparation comprising a compound of claim 1 and a pharmaceutical carrier.

23. A method of promoting anti-tumor activity in a mammalian subject, comprising administering a effective amount of a compound of claim 1 and a pharmaceutical carrier.

24. A method for preventing carcinogenesis comprising: administering to a mammalian subject having precancerous precursors, a pharmaceutical preparation comprising a compound of claim 1 and a pharmaceutically acceptable salt in an amount effective to prevent the occurrence of the cancer or precancerous condition or to slow, halt or reverse the progression of the cancer or precancerous condition.

25. A method for treating carcinogenesis comprising: administering to a mammalian subject having precancerous precursors, a pharmaceutical preparation comprising a compound of claim 1 and a pharmaceutically acceptable salt in an amount effective to prevent the occurrence of the cancer or precancerous condition or to slow, halt or reverse the progression of the cancer or precancerous condition.

26. A method of preventing the occurrence or progression of a cancer or a precancerous condition in a mammal comprising administering to the mammal an effective amount of a composition comprising a compound of claim 1 and a pharmaceutically acceptable carrier.

27. The method of claim 26, wherein the composition is administered prophylactically to prevent the occurrence of the cancer or precancerous condition before, during, or after exposure of the mammal to a known or suspected carcinogenic or procarcinogenic compound, agent, or event.

28. A method of treating inflammation, comprising administering an effective amount of a compound of claim 1 and a pharmaceutically acceptable carrier.

29. A method of treating or preventing cancer in a mammalian subject, comprising administering an effective amount to the mammalian subject a pharmaceutical preparation comprising a chemopreventive or chemotherapeutic compound of the following formula: 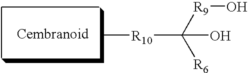
wherein
R₆ is H, or a substituted or unsubstituted alkyl group; R₉ is a bond or a substituted or unsubstituted alkyl group; R₁₀ is a bond or a substituted or unsubstituted alkyl group; and the cembranoid compound is substituted or unsubstituted.
